(19) United States
(12) Patent Application Publication
(10) Pub. No.: US 2014/0221961 A1
(43) Pub. Date: Aug. 7, 2014

(54) INSERTABLE MEDICAL DEVICE FOR DELIVERING NANO-CARRIERS OF MITOMYCIN (AND ITS ANALOGUES) TO A TARGET SITE, AND METHODS FOR PREPARING AND USING THE SAME

(71) Applicant: Vascular Nanotransfer Technologies LLC, Chatham, NJ (US)

(72) Inventor: Juan Granada, Upper Saddle River, NJ (US)

(21) Appl. No.: 14/175,758

(22) Filed: Feb. 7, 2014

Related U.S. Application Data
(60) Provisional application No. 61/761,986, filed on Feb. 7, 2013.

(51) Int. Cl. A61L 29/16; A61L 31/16; A61L 29/08; A61K 31/407; A61K 9/51

(52) U.S. Cl. CPC A61L 29/16; A61K 31/407; A61K 9/5123; A61L 29/08; A61L 31/16
USPC 604/500; 514/410; 424/423; 424/490; 424/498; 623/1.11; 604/93.01; 264/4

(57) ABSTRACT

A nano-carrier comprising an antiproliferative drug encapsulated by a lipophilic enhancement agent.

### REFERENCE TO PENDING PRIOR PATENT APPLICATION

This patent application claims benefit of pending prior U.S. Provisional Patent Application Ser. No. 61/761,986, filed Feb. 7, 2013 by Juan Granada for INSERTABLE MEDICAL DEVICES FOR DELIVERING NANO-CARRIERS OF MITOMYCIN TO A TARGET SITE AND METHODS FOR PREPARING THE SAME (Attorney's Docket No. VNT-1 PROV), which patent application is hereby incorporated herein by reference.

### FIELD OF THE INVENTION

This invention relates to insertable medical devices for delivering therapeutic agents into the tissue of diseased human organs. More particularly, this invention relates to insertable medical devices coated with nano-carriers of one or more therapeutic agents for delivering those therapeutic agents into a diseased human artery or other human tissues. Even more particularly, this invention relates to methods for preparing nano-carriers of Mitomycin (and its analogues) so as to increase the lipophilic properties of the Mitomycin (and its analogues), and to methods for mounting the nano-carriers of Mitomycin (and its analogues) to the insertable medical devices and using the same to treat a patient.

### BACKGROUND OF THE INVENTION

Since the early stages of percutaneous vascular intervention, controlling restenosis has been an important measure of success, and a great deal of effort has gone into understanding its mechanisms. Restenosis is the result of a variety of mechanical and biological processes which, in balloon angioplasty procedures, typically begin immediately following balloon deployment, including acute vessel recoil, negative vascular remodeling and excessive neointimal proliferation.

The most severe side effect of balloon angioplasty, i.e., abrupt vessel closure resulting from elastic recoil and plaque dissection, was effectively solved by the introduction of balloon-expandable stents. However, even with balloon-expandable stents, in-stent restenosis, caused by excessive neointimal proliferation, can still occur. Fortunately, drug-eluting stents (DES) have recently been developed which effectively reduce in-stent restenosis by eluting an antiproliferative drug and thereby reducing neointimal proliferation. As a result, drug-eluting stents (DES) have now become a mainstay in the treatment of coronary atherosclerotic disease.

However, the demonstrated efficacy of drug-eluting stents (DES) in coronary intervention is still balanced by the small but real (and unpredictable) risk of very late stent thrombosis, which is believed to be due to delayed vascular healing as a result of (i) the initial antiproliferative effect (and associated late, incomplete stent apposition), or (ii) a hypersensitivity to the antiproliferative drug which is eluted by the stent to control excessive neointimal proliferation, or (iii) a hypersensitivity to the polymer coating which is added to the stent for carrying and releasing the antiproliferative drug, or (iv) combinations of the above.

In recent years, drug-coated balloons (DCB) have emerged as a therapeutic alternative in the interventional field. With this technology, short-term transfer of antiproliferative drugs to the arterial wall is thought to be achieved, thus potentially reducing the negative effects associated with the longer-term drug release pattern commonly associated with polymer-based drug-eluting stent (DES) technologies. In small clinical trials, Paclitaxel-coated balloons (PCB) were shown to be safe and effective in reducing restenosis among patients with coronary in-stent restenosis and de novo peripheral vascular disease lesions. However, the long term safety and efficacy of these drug-coated balloon (DCB) and Paclitaxel-coated balloon (PCB) technologies, particularly with respect to integrating this technology with currently-approved technologies such as stents and atherectomy, and/or integrating these technologies into new clinical applications (e.g., small vessels, bifurcations, etc.), is still under investigation.

The concept of delivering drugs into the vessel wall through a single-time dose application during an angioplasty procedure for the prevention of restenosis has been present for almost 20 years. However, despite extensive efforts to improve the efficiency of local arterial delivery, several studies have shown a marked variability in the site-specific uptake of the drugs and a rapid clearance of the delivered compounds, thus discouraging the widespread use of these technologies. In addition, the successful development of easy-to-use balloon-expandable stents has superseded the development of clinically functional local drug delivery technologies and the development of drugs offering an acceptable efficacy profile, thus heightening the general skepticism as to the potential commercialization of this technology.

Several biological, technical and clinical aspects make balloon-based local drug delivery an attractive alternative to current drug-eluting stent (DES) technologies for the treatment of atherosclerotic vascular disease. First, in contrast to the clinical situation encountered by researchers and clinicians in the past, several antiproliferative agents with a successful record of clinical safety and efficacy are now clinically available. Second, the theoretical advantages which are associated with the homogeneous and uniform drug delivery that can be achieved using drug-coated balloons (DCB) may lead to greater therapeutic safety and efficacy. Third, the lack of an ongoing presence of both an antiproliferative drug and/or an irritating polymer coating on the stent (for carrying and releasing the antiproliferative drug) may lead to more rapid vascular healing, thus reducing any inflammation due to a hypersensitivity to those elements and resulting in a shorter time requirement for dual anti-platelet therapy.

In order to avoid the side effects associated with the polymers used in polymer-based stent technologies, several non-polymeric approaches have also been cited in the literature. These non-polymeric approaches use microparticles or nanoparticles of the antiproliferative drugs deposited on the surface of the drug-coated balloons (DCBs). These antiproliferative drugs are then transferred from the drug-coated balloons (DCBs) to the vessel walls when the balloon is inflated. However, these non-polymeric approaches are generally based on modifying the surface of the balloons in order to provide depot effects for the antiproliferative drug. These surface modification approaches do not guarantee the uniform distribution of the microparticles or nanoparticles of the antiproliferative drugs across the treated area, thus negatively impacting the process of healing the treated area. Furthermore, such non-polymeric approaches have generally taken advantage of the chemical characteristics of highly lipophilic drugs such as Paclitaxel. In contrast, less lipophilic drugs such as Sirolimus and Mitomycin have not been successfully loaded onto the surfaces of medical devices unless controlled polymer-based delivery systems are used. Of course, such polymer-based delivering systems in turn introduce the hypersensitivity issues discussed above. As a result, it has heretofore been impractical to deliver less lipophilic drugs (e.g., Sirolimus and Mitomycin) using inflatable balloons.

With advances in the use of nano-technology in the field of medicine, the development of nano-sized particles has gained importance in the field of local drug delivery. Generally, polymers are used to construct nano-sized particles containing the drugs and acting as local drug delivery agents. However, the use of a polymer as a drug delivery agent brings additional challenges to the various processes of drug transfer, drug release and long-term drug retention. In addition, these polymer-based nano-particles may induce inflammation in tissue due to the degradation process of the polymer at the treatment site. Furthermore, due to the degradation properties of the polymer, the antiproliferative drug may be delivered in an unpredictable manner, thus resulting in non-homogeneous healing (“edge effect and delayed healing”). In addition, drugs displaying different solubility profiles, such as Mitomycin, require further modification to enhance long term retention on, and absorption into, the arterial tissue.

Thus there is a need in the art for improving the delivery and uptake of Mitomycin (and its analogues) and/or other antiproliferative drugs by preparing nano-carriers, utilizing an encapsulation matrix, so as to improve the lipophilic property of the Mitomycin (and its analogues) and/or other antiproliferative drugs. There is also a need in the art for an insertable medical device which can carry and deliver the Mitomycin (and its analogues) and/or other antiproliferative drugs (in the form of encapsulated nano-carriers) without requiring the physical surface of the insertable medical device to be modified. Alternatively, there is a need in the art for enhancing the release of the aforementioned drug nano-carriers by applying a release-promoting layer on the surface of the insertable medical device (e.g., on the surface of the balloons) so as to promote the release of the nano-carriers from the surface of the balloons. Additionally, there is a need in the art to facilitate the delivery and uptake of the Mitomycin (and its analogues) and/or other antiproliferative drugs without requiring the use of polymers.

### SUMMARY OF THE INVENTION

The present invention provides for the improved delivery and uptake of Mitomycin (and its analogues) and/or other antiproliferative drugs by preparing nano-carriers, utilizing an encapsulation matrix, so as to improve the lipophilic property of the Mitomycin (and its analogues) and/or other antiproliferative drugs. The present invention also provides an insertable medical device which can carry and deliver the Mitomycin (and its analogues) and/or other antiproliferative drugs (in the form of encapsulated nano-carriers) without requiring the physical surface of the insertable medical device to be modified. Alternatively, the present invention also provides for the enhanced release of the aforementioned drug nano-carriers by applying a release-promoting layer on the surface of the insertable medical device (e.g., on the surface of the balloons) so as to promote the release of the nano-carriers from the surface of the balloons. And the present invention provides novel methods and apparatus to facilitate the delivery and uptake of the Mitomycin (and its analogues) and/or other antiproliferative drugs without requiring the use of polymers.

In one preferred form of the present invention, there is provided a nano-carrier comprising an antiproliferative drug encapsulated by a lipophilic enhancement agent.

In another preferred form of the present invention, there is provided a method for forming nano-carriers, the method comprising:

providing a solution of an antiproliferative drug and providing a solution of a lipophilic enhancement agent;

combining the solution of the antiproliferative drug and the solution of the lipophilic enhancement agent so as to provide a solution of nano-carriers comprising the antiproliferative drug encapsulated by the lipophilic enhancement agent.

In another preferred form of the present invention, there is provided apparatus for delivering nano-carriers to a patient, the apparatus comprising:

an insertable medical device; and

nano-carriers disposed on the outer surface of the insertable medical device;

wherein the nano-carriers comprise an antiproliferative drug encapsulated by a lipophilic enhancement agent.

In another preferred form of the present invention, there is provided a method for treating a patient, the method comprising:

providing a nano-carrier comprising an antiproliferative drug encapsulated by a lipophilic enhancement agent; and

administering the nano-carrier to the patient.

### BRIEF DESCRIPTION OF THE DRAWINGS

These and other objects and features of the present invention will be more fully disclosed or rendered obvious by the following detailed description of the preferred embodiments of the invention, which is to be considered together with the accompanying drawings wherein like numbers refer to like parts, and further wherein:

FIG. 1 is a schematic view showing how encapsulated nano-carriers may be produced and mounted on an insertable medical device; and

FIG. 2 is a schematic view showing the chemical structure of Mitomycin C.

### DETAILED DESCRIPTION OF THE PREFERRED EMBODIMENTS

Before describing in detail preferred embodiments that are provided in accordance with the present invention, it should be observed that the embodiments reside primarily in the combination of a drug-releasing insertable medical device coated with encapsulated nano-carriers of an antiproliferative drug. Accordingly, the various components and method steps described herein include only those specific details that are pertinent to understanding the embodiments of the present invention so as to not obscure the disclosure with details that will be readily apparent to those of ordinary skill in the art having the benefit of the description contained herein.

In this document, the terms “comprises”, “comprising” and/or any other variation thereof are intended to constitute a non-exclusive inclusion, such that a process, method, article or apparatus that “comprises” a list of elements includes not only those elements recited, but may also include other elements not expressly stated or inherent to such process, method, article or apparatus. Furthermore, an element proceeded by “comprises . . . a” does not, without additional constraints, preclude the existence of additional identical elements in the process, method, article or apparatus that comprises the element.

In addition, before describing in detail the various preferred embodiments that are provided in accordance with the present invention, it should be observed that the scientific and technical terms used herein for describing the invention have the same meanings as would be commonly understood by a person skilled in the art.

In the following description, specific embodiments of the invention are described. However, one of ordinary skill in the art will appreciate that various modifications and changes may be made to the specific embodiments disclosed herein without departing from the spirit and scope of the present invention. Accordingly, the specification is to be regarded in an illustrative, rather than a restrictive, sense and all such modifications are intended to be included within the scope of the present invention.

Generally speaking, pursuant to various embodiments, the invention comprises the provision and use of novel nano-carriers of an antiproliferative drug (e.g., Mitomycin and its analogues), methods for preparing the nano-carriers of the antiproliferative drug, methods for preparing a drug-releasing insertable medical device (DRIMD) for delivering those nano-carriers of the antiproliferative drug (and hence delivering the antiproliferative drug itself) to a target site within a body lumen, and methods for using the insertable medical device to provide therapy to a patient.

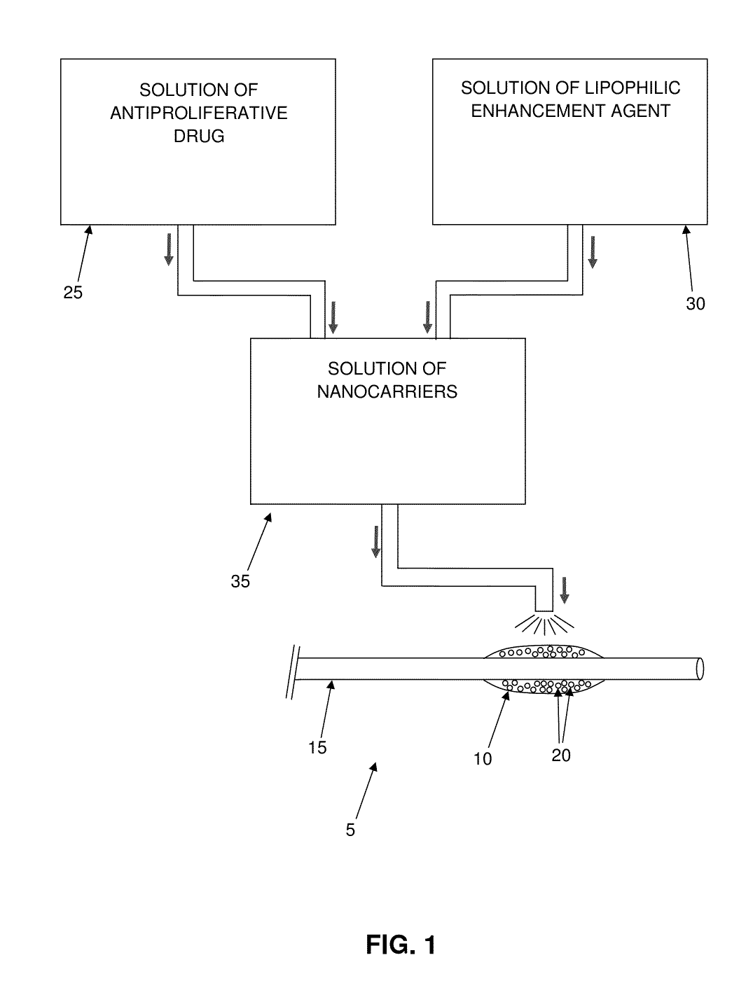

In accordance with the present invention, there is provided a drug-releasing insertable medical device (DRIMD) 5 (FIG. 1) which generally comprises a balloon 10 (and/or a stent) and a delivery catheter 15 carrying the balloon 10 (and/or the stent). Encapsulated nano-carriers 20, carrying one or more drugs to prevent restenosis, are applied to the surface of the drug-releasing insertable medical device (DRIMD) 5, e.g., to the surface of the balloon 10. If desired, the release of the encapsulated nano-carriers 20 may be enhanced by providing a release-promoting layer (not shown) on the surface of the drug-releasing insertable medical device (DRIMD) 5.

In one form of the present invention, the drug-releasing insertable medical device (DRIMD) 5 comprises a balloon catheter and/or a stent assembly generally used for Percutaneous Transluminal Angioplasty (PTA). For example, a PTA catheter may be an angioplasty balloon catheter. In one preferred embodiment, the drug-releasing insertable medical device (DRIMD) 5 is a balloon catheter assembly generally used for Percutaneous Transluminal Coronary Angioplasty (PTCA). The balloon catheter assembly essentially includes a balloon catheter 15 and an inflatable balloon 10 mounted on the balloon catheter 15. Additionally, the balloon catheter assembly contains additional functioning elements for the intended application (e.g., lumens for inflating or deflating the balloons, etc.), and the invention primarily resides in the inflatable balloon section, coated with the nano-carriers of one or more drugs. The functioning and the components comprising the balloon catheter assembly (other than the drug-coated inflatable balloon) are not disclosed in detail herein, since they are well known in the art and do not constitute the novel aspects of the present invention.

In yet another embodiment, the drug-releasing insertable medical device (DRIMD) 5 comprises a stent (not shown) mounted on an expandable balloon. The nano-carriers 20 of the antiproliferative drug are positioned as a layer on the outer surface of the stent and on the outer surface of the balloon. In this construction, both the stent and balloon serve to carry and release the nano-carriers 20 (and hence, the antiproliferative drug).

The tissue to which the nano-carriers of the antiproliferative drug is directed may comprise a blood vessel, the urethra, the esophagus, the bile duct, etc. The drug-releasing insertable medical device (DRIMD) 5 may be used to treat a medical condition associated with the narrowing of a body lumen. By way of example but not limitation, the medical condition may be a blocked artery, esophageal narrowing or other type of visceral stenosis.

The preparation process for producing the encapsulated nano-carriers 20 includes the preparation of a solution 25 (FIG. 1) of the antiproliferative drug and the preparation of a solution 30 of a lipophilic enhancement agent. It should be appreciated that the solution 30 of the lipophilic enhancement agent preferably further comprises a surfactant. The antiproliferative drug (e.g., Mitomycin or its analogues) is converted into nano form in its solution 25; and the lipophilic agent is converted into nano form in its solution 30. The nano preparation process includes high speed homogenization, such as by ultrasound homogenizer, high pressure homogenizer, etc. The nano solution 30 of the lipophilic enhancement agent is combined with the solution 25 of the drug nano-crystals and together they produce a solution 35 which may then be sprayed on the inflatable balloon 10 (or other surface area) of the drug-releasing insertable medical device (DRIMD) 5. This solution 35 provides the encapsulated nano-carriers 20 of the antiproliferative drug (i.e., the lipophilic enhancement agent encapsulates the antiproliferative drug). Thus, the lipophilic agent is essentially a drug carrier which carries the antiproliferative drug (e.g., the Mitomycin or its analogues) inside of the lipophilic enhancement agent.

Alternatively, the nano-carriers 20 can be prepared in Mitomycin-phosphoramidite complex form, converted into nano form and then encapsulated with encapsulating agents.

In one embodiment of the present invention, the drug carrier (i.e., the coating of the lipophilic enhancement agent) comprises a single excipient. In another embodiment of the present invention, the drug carrier may comprise a plurality of excipients. Those skilled in the art will appreciate that one or more releasing carriers may be used with the present invention. In some cases, the releasing carrier which is used as an encapsulating medium may also have a desirable effect on the antiproliferative drug which is being encapsulated. By way of example but not limitation, the encapsulating medium may enhance the lipophilicity of the antiproliferative drug so as to increase affinity for the tissue, and/or the encapsulating medium may enhance the emulsifying properties of the antiproliferative drug so as to increase tissue transfer, and/or the encapsulating medium may enhance the stabilization properties of the nano-carrier so as to reduce the potential for drug toxicity. The encapsulating agent may be one or more of a biologically-derived excipient, phosphorus-containing essential lipid, and/or a phosphoramdite group-containing compound. The antiproliferative drug is surrounded by the encapsulating agent, i.e., the antiproliferative drug sits inside the encapsulation medium, so that the antiproliferative drug forms the inner core of the nano-carrier, with the lipophilic enhancement agent forming the encapsulation medium of the nano-carrier.

In one preferred form of the invention, the drug-releasing insertable medical device (DRIMD) 5 includes a balloon 10 mounted on a balloon catheter 15. One or more parts of the balloon catheter surface are coated with the nano-carriers 20 of one or more antiproliferative drugs. When the inflatable part of the balloon 10 comes into contact with the surrounding wall of the body lumen, the nano-carriers 20 of the drug are transferred to the lumen (e.g., arterial) wall, ranging from about 10% to 90% of the total gravimetric load within about 10 to 90 seconds of balloon inflation time.

Optionally, the inflatable balloon 10 (and/or other portions of the balloon catheter 15) may have a release-promoting layer (not shown) formed on the outer surface of the balloon 10 (and/or other portions of the balloon catheter 15) so as to enhance release of the nano-carriers 20. This release-promoting layer induces delivery of the nano-carriers 20 to the wall of the body lumen. The drug-releasing insertable medical device (DRIMD) 5 preferably has such a release-promoting layer in at least a circumferential configuration and folded configuration so as to provide uniform distribution of the nano-carriers upon nominal balloon expansion.

According to various embodiments, in addition to the aforementioned optional release-promoting layer (provided on the surface of the balloon), the drug-releasing insertable medical device (DRIMD) 5 may also have one or more uncoated surfaces in a random position on the drug-releasing insertable medical device (DRIMD) 5. The one or more uncoated surfaces may be defined as locations on the drug-releasing insertable medical device (DRIMD) 5 where nano-carriers are not deposited.

Thus, in one preferred form of the present invention, the inflatable part of the balloon catheter comprises a nano-carrier coating containing nano-carriers of the antiproliferative drug deposited on a release-promoting surface of the balloon catheter 15, either randomly or in a desired pattern.

The encapsulated nano-carrier 20 comprises one or more antiproliferative drugs surrounded by an encapsulating medium (e.g., a lipophilic enhancement agent). The antiproliferative drug may include nano-crystals of the antiproliferative drug. The nano-crystals of the antiproliferative drug may have a z-average diameter ranging from about 1 nm to about 5000 nm. Furthermore, the nano-crystals of the antiproliferative drug may have two or more different average diameters. Alternatively, the antiproliferative drug may include one or more of nano-sized particles, nano-spheres, liposomes, nano-capsules, dendrimers and/or any other form of the drug that has nano-dimensions.

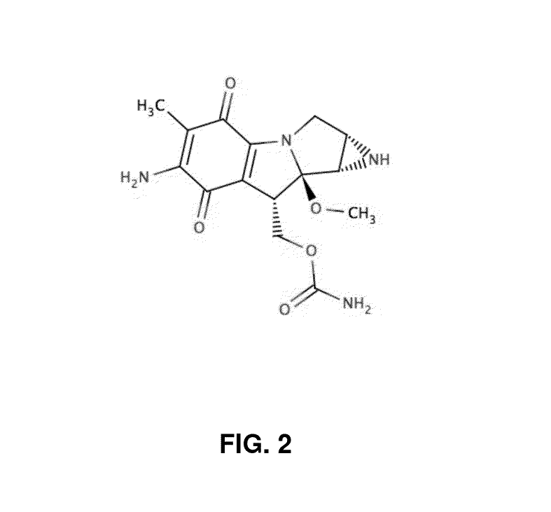

In one preferred embodiment of the present invention, the antiproliferative drug is Mitomycin C. The chemical formula of Mitomycin C is C₁₅H₁₈N₄O₅ and the chemical structure of Mitomycin C is shown in FIG. 2.

In another embodiment of the present invention, the Mitomycin C is an analogue related to the quinone-containing alkylating agents of the Mitomycin family having antiproliferative and antibiotic properties. In the present invention, the quinone-containing alkylating agents may include, but are not limited to, Mitomycin A, Mitomycin A analogue 7-(2-hydroxyethoxy) mitosane (BMY 2551), Mitomycin B, Mitomycin C, KW-2149, BMS-191174, BMY 25282, BMY 25067, MC-77, MC-62, porfiromycin, acetylmitomycin C, FR-900482, FR-66979, FK-973, and combinations of the above. Those skilled in the art will recognize there are other derivatives, substitutes or analogues related to the Mitomycin family known in the art which are within the spirit and scope of the present invention. They may all sometimes be referred to herein under the general terms “Mitomycin” and/or “Mitomycin (and its analogues)”.

Mitomycin C causes inhibition of smooth muscle cell proliferation in an anaerobic (low oxygen) environment. The present invention provides an effective method for treating a localized area of a diseased vasculature by means of a drug-releasing insertable medical device (DRIMD) that comprises a coating which elutes Mitomycin C at a controlled rate and inhibits the proliferation of smooth muscle cells that cause restenosis, is reliable in consistently treating the localized area over a period of time and does not adversely affect healthy tissue adjacent to the area of treatment. After delivery of the encapsulated nano-carriers to the target site, the antiproliferative drug is transferred to the inner surface of the body lumen according to a concentration gradient differential. More particularly, the dissolution of the encapsulating medium leads to delivery of the antiproliferative drug and thereby provides beneficial biological effects to the tissue. In addition, Mitomycin inhibits DNA synthesis. Mitomycin is a chemotherapeutic antibiotic used for some types of cancer. Mitomycin effectively inhibits in-vitro proliferation of smooth muscle cells at various concentrations without adverse effects to a patient. Mitomycin is cytostatic at certain dosages and cytotoxic at different dosages. Therefore, a person skilled in the art may design a drug-releasing insertable medical device (DRIMD) which provides the desired optimal drug dose for the patient.

In one embodiment of the present invention, the optional release-promoting layer deposited on the balloon catheter (for promoting release of the nano-carriers) may include, by way of example but not limitation, gelatin, gluten, albumin, water soluble vitamins, fat soluble vitamins, fatty acids, bile acids and fatty esters.

Furthermore, in one embodiment of the present invention, the encapsulating agent (i.e., the lipophilic enhancement agent) may include, by way of example but not limitation, steroids, esterified fatty acids, phosphatidylcholine, phosphatidylglycerol, phosphatidylinositol, phosphatidylserine, phosphatidic acid, cardiolipin, and phosphatidylethanolamine, a compound containing phoshoramidite group contains at least a Cholesteryl-TEG Phosphoramidite, 5′-Cholesteryl-TEG Phosphoramidit, α-Tocopherol-TEG Phosphoramidite and 5′-Stearyl Phosphoramidite.

The drug-releasing insertable medical device (DRIMD), by releasing nano-carriers of a antiproliferative drug, inhibits restenosis and associated complications.

In one form of the present invention, there is provided a method for preventing restenosis, comprising: providing a biocompatible insertable medical device; applying encapsulated nano-carriers, comprising an antiproliferative drug (e.g., an analogue related to the quinone-containing alkylating agents of the Mitomycin family) encapsulated by a lipophilic enhancement agent, to the medical device; and inserting the medical device into a vessel to elute nano-carriers of the antiproliferative drug (e.g., quinone-containing alkylating agents of the Mitomycin family) to the surrounding tissue.

In another form of the invention, there is provided a method for treating restenosis, comprising: providing a biocompatible insertable medical device; preparing encapsulated nano-carriers of an antiproliferative drug; co-solubilizing encapsulated nano-carriers with a solvent to form a drug coating solution; optionally applying a release-promoting layer on the insertable medical device; applying the drug coating solution to the insertable medical device to form an elution layer comprising the encapsulated nano-carriers of the antiproliferative drug; allowing the solvent to evaporate; and inserting the medical device into a body for delivering encapsulated nano-carriers of the antiproliferative drug at the target site.

The antiproliferative drug present in the nano-carriers is preferably deposited on an inflatable portion of the balloon that is exposed to the body lumen (e.g., to a blood vessel). The antiproliferative drug releases upon dissolution of the encapsulating lipophilic enhancement agent. The nano-carriers from which the antiproliferative drug is released provide a therapeutic drug to different layers of the tissue (e.g., to different layers of an artery). Thus, because of the in-tissue release of the antiproliferative drug and the in-tissue diffusion of the antiproliferative drug, the antiproliferative drug may be delivered to the maximum amount of a lesion at the target site. The total drug-to-drug carrier ratio may include, by way of example but not limitation, 1 part of drug to 99 parts of drug carrier, or vice versa. For example, a 10:90 drug-to-drug carrier ratio includes 10 parts of drug and 90 parts of drug carrier.

In one embodiment of the present invention, a total dosage of about 0.1-50 micrograms of the antiproliferative drug is provided per square millimeter of surface area of the drug-releasing insertable medical device (DRIMD). Those skilled in the art will recognize that the dosage of the antiproliferative drug per millimeter of a length of the biocompatible implantable medical device can vary and be within the spirit and scope of the present invention.

### Modifications

It will be appreciated that further embodiments of the present invention will be apparent to those skilled in the art in view of the present disclosure. It is to be understood that the present invention is by no means limited to the particular constructions herein disclosed and/or shown in the drawings, but also comprises any modifications or equivalents within the scope of the invention.

What is claimed is:

1. A nano-carrier comprising an antiproliferative drug encapsulated by a lipophilic enhancement agent.

2. A nano-carrier according to claim 1 wherein the antiproliferative drug is selected from the group consisting of Mytomycin and its analogues.

3. A nano-carrier according to claim 1 wherein the antiproliferative drug comprises Mytomycin C.

4. A nano-carrier according to claim 1 wherein the antiproliferative drug is selected from the group consisting of Mitomycin A, Mitomycin A analogue 7-(2-hydroxyethoxy) mitosane (BMY 2551), Mitomycin B, Mitomycin C, KW-2149, BMS-191174, BMY 25282, BMY 25067, MC-77, MC-62, porfiromycin, acetylmitomycin C, FR-900482, FR-66979, FK-973, and combinations thereof.

5. A nano-carrier according to claim 1 wherein the lipophilic enhancement agent is selected from the group consisting of steroids, esterified fatty acids, phosphatidylcholine, phosphatidylglycerol, phosphatidylinositol, phosphatidylserine, phosphatidic acid, cardiolipin, and phosphatidylethanolamine, a compound containing phoshoramidite group contains at least a Cholesteryl-TEG Phosphoramidite, 5′-Cholesteryl-TEG Phosphoramidit, α-Tocopherol-TEG Phosphoramidite and 5′-Stearyl Phosphoramidite.

6. A method for forming nano-carriers, the method comprising:
providing a solution of an antiproliferative drug and providing a solution of a lipophilic enhancement agent;
combining the solution of the antiproliferative drug and the solution of the lipophilic enhancement agent so as to provide a solution of nano-carriers comprising the antiproliferative drug encapsulated by the lipophilic enhancement agent.

7. A method according to claim 6 further comprising creating solid nano-carriers from the solution of nano-carriers.

8. Apparatus for delivering nano-carriers to a patient, the apparatus comprising:
an insertable medical device; and
nano-carriers disposed on the outer surface of the insertable medical device;
wherein the nano-carriers comprise an antiproliferative drug encapsulated by a lipophilic enhancement agent.

9. Apparatus according to claim 8 wherein the insertable medical device comprises a catheter.

10. Apparatus according to claim 8 wherein the insertable medical device comprises a balloon catheter.

11. Apparatus according to claim 8 wherein the insertable medical device comprises a stent.

12. Apparatus according to claim 11 wherein the insertable medical device comprises a stent mounted on a balloon catheter.

13. Apparatus according to claim 8 wherein the antiproliferative drug is selected from the group consisting of Mytomycin and its analogues.

14. Apparatus according to claim 8 wherein the outer surface of the insertable medical device comprises a release-promoting layer, and further wherein the nano-carriers are disposed on the release-promoting layer.

15. Apparatus according to claim 15 wherein the release-promoting layer is formed from at least one material selected from the group consisting of gelatin, gluten, albumin, water soluble vitamins, fat soluble vitamins, fatty acids, bile acids and fatty esters.

16. A method for treating a patient, the method comprising:
providing a nano-carrier comprising an antiproliferative drug encapsulated by a lipophilic enhancement agent; and
administering the nano-carrier to the patient.

17. A method according to claim 16 wherein the nano-carrier is disposed on an insertable medical device, and further wherein the step of administering the nano-carrier to the patient comprises inserting the insertable medical device into the patient.

18. A method according to claim 17 wherein the antiproliferative drug is selected from the group consisting of Mytomycin and its analogues.

19. A method according to claim 17 wherein the insertable medical device comprises a balloon catheter.

20. A method according to claim 19 wherein the nano-carrier is disposed on the balloon of the balloon catheter, and further wherein the step of administering the nano-carrier to the patient comprises inflating the balloon so as to bring the nano-carrier into contact with the tissue of the patient.
